(19) United States
(12) Plant Patent Application Publication
(10) Pub. No.: US 2017/0112037 P1
(43) Pub. Date: Apr. 20, 2017

(54) Lachenalia Jacq. f. ex Murray named ''Aqua Lady''

(50) ;

(71) Applicant: AGRICULTURAL RESEARCH COUNCIL, Pretoria (ZA)

(72) Inventor: Riana Kleynhans, Benoni (ZA)

(21) Appl. No.: 14/756,803

(22) Filed: Oct. 15, 2015

(51) Int. Cl. A01H 5/00

(52) U.S. Cl. PLT/263.1

(57) ABSTRACT

A new and distinct form of Lachenalia plant characterized by a bulb plant type having a spreading growth habit of a height of 15-30 centimetres (cm) and width of 10-15 cm having a turquoise blue flower and green leave with maroon spots, further characterized in that the spots are relatively small in size and fairly pronounced in that it has a good contrast.

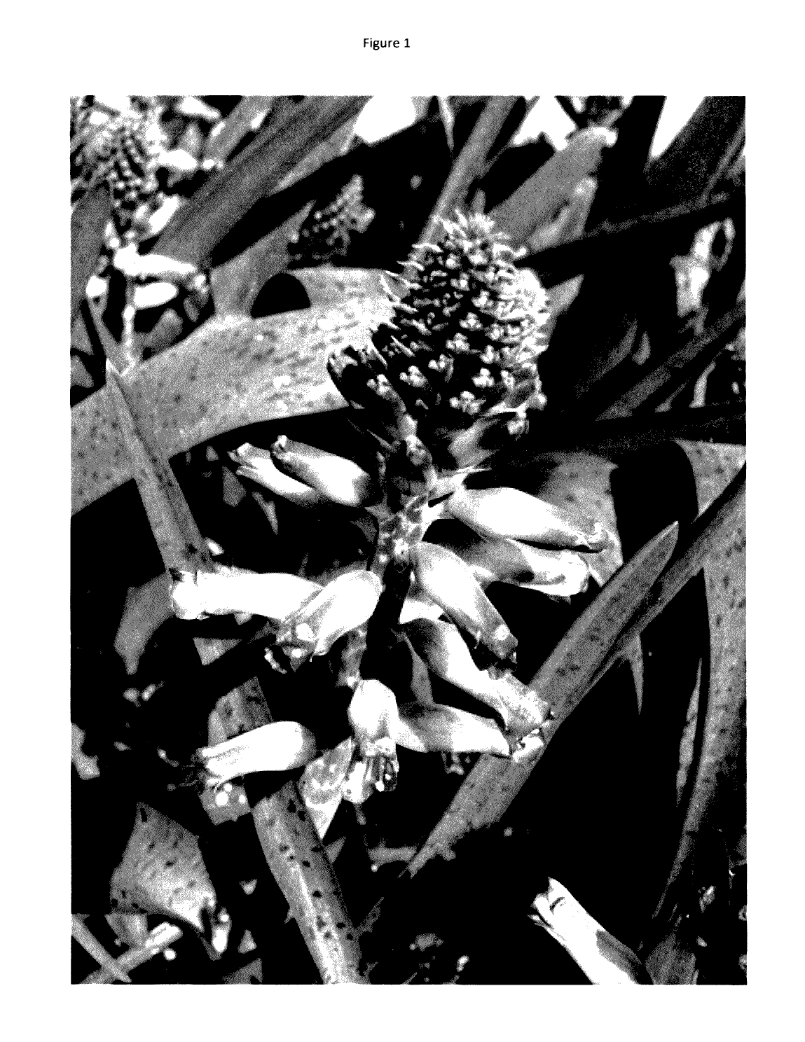

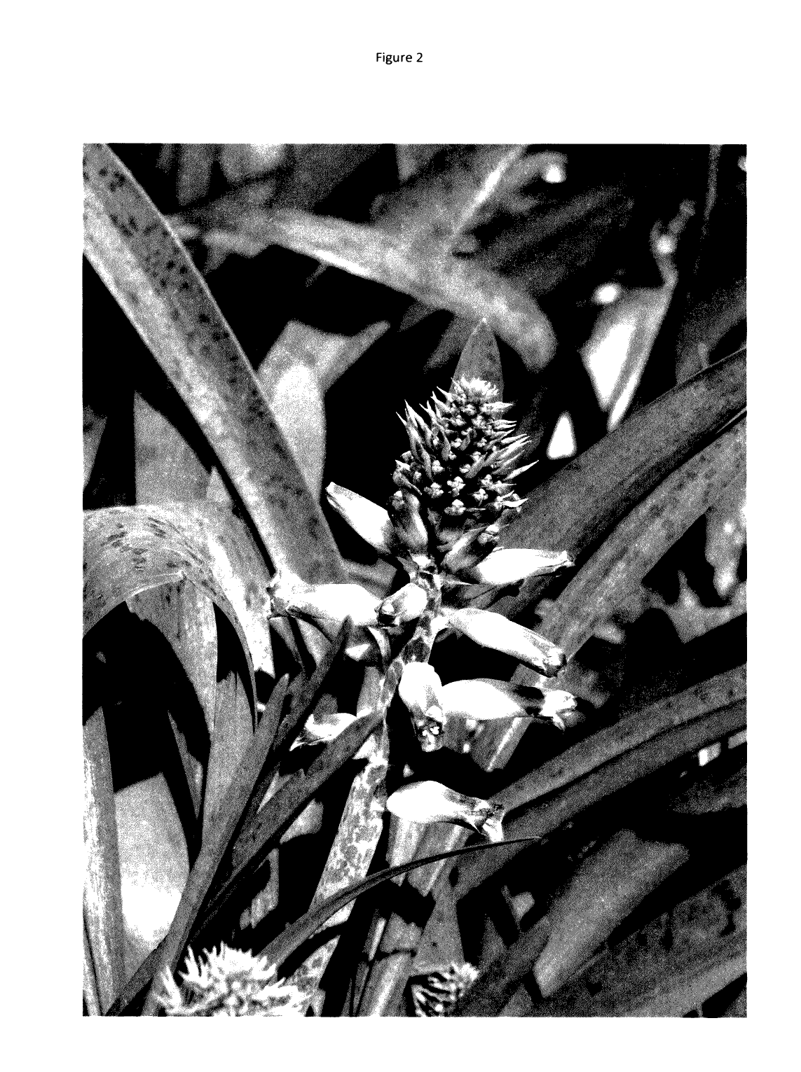

Genus and species: Lachenalia Jacq. f. ex Murray.

Variety denomination: Aqua Lady.

### BACKGROUND OF THE INVENTION

The present invention relates to a new and distinct cultivar of Lachenalia, botanically known as Lachenalia Jacq. f. ex Murray having the common name of Viooltjies/Cape cowslip and hereinafter referred to by its variety denomination “Aqua Lady”.

The variety was first cultivated in 2008 and originated by means of conventional breeding by the inventor during 2009 to 2010 at the premises of Agricultural Research Council Vegetable and Ornamental Plant Institute, Roodeplaat, Pretoria, Republic of South Africa.

Asexual reproduction of the Aqua Lady plant has shown that the unique features thereof are stable and reproduced true to type in successive generations.

### SUMMARY OF THE INVENTION

The present invention constitutes a new and distinct variety of Lachenalia which originated from the controlled pollination between a Lachenalia hybrid as seed bearing parent and a Lachenalia viridiflora as pollen parent as detailed hereinbelow.

The following traits have been repeatedly observed and are determined to be the unique characteristics, under the local evaluation conditions, of the Aqua Lady plant. These characteristics in combination distinguish Aqua Lady as a new and distinct Lachenalia cultivar:
1. Bulb plant type having a spreading growth habit of a height of 15-30 centimetres (cm) and width of 10-15 cm.
2. Flower color: turquoise blue.
3. Leaf color: green with maroon spots.

Plants of the new Lachenalia variety Aqua Lady can be compared to the variety denomination Rupert but differs in that the flower color of Aqua Lady is turquoise compared to the purple of Rupert. The maroon spots of the Aqua Lady plant is furthermore smaller in size, yet more pronounced in that it has a good contrast compared to the low contrast found in Rupert.

### BRIEF DESCRIPTION OF THE PHOTOGRAPHS

The accompanying colored photographs illustrate the overall appearance of the Aqua Lady, showing the colors as true as it is reasonably possible to obtain in colored reproductions of this type. The photographs comprise a front perspective view of a typical Aqua Lady grown in an indoor nursery, but located in an outdoor setting.

### DETAILED DESCRIPTION

The aforementioned photographs, following observations and measurements describe Aqua Lady grown by the inventor on behalf of Agricultural Research Council Vegetable and Ornamental Plant Institute, Roodeplaat, Pretoria, Republic of South Africa. Thereafter Aqua Lady was made subject of a South African Plant Breeders' Right application in March 2009. This application was granted in 2010 under number ZA20104392.

The plant is an early flowering cultivar and may be used for landscaping and should be planted during the month of March; indoors and in pots only. The plant should be kept at temperatures of 20-25 degrees Celsius during the day and 10-15 degrees Celsius at night. Plenty of light should be provided.

Aqua Lady Is a hybrid-hybrid cross containing the following Lachenalia species: Lachenalia viridiflora, Lachenalia orchioides subsp. glaucina, Lachenalia bulbifera, Lachenalia quadricolor and Lachenalia rubida.
Leaf: Monocolored.
   Spots on upper side.—Present, small and with good contrast.
   Attitude.—Semi-erect.
   Length.—Medium — 22.5 cm.
   Width.—Medium — 4.8 cm.
   Ratio.—length/width Large (elongated).
   Shape.—Oblong.
   Color.—Dark green.
   Shape in cross section.—Angular.
   Recurving and undulation of margin.—Absent.
   Blistering and pubescence of upper side.—Absent.
   Spots on upper side.—Present (Purple).
   Size of spots on upper side.—Small to medium.
   Density of spots on upper side.—Medium.
   Intensity of spots on upper side.—Dark.
   Markings on outer side of leaf base.—Present.
Peduncle:
   Length.—Medium.
   Spots or markings.—Present.
   Size of spots or markings.—Large.
   Density of spots or markings.—Dense.
Interflorescense:
   Length.—Medium.
   Rudimentary apex.—Conspicuous.
   Number of flowers.—Few.
Flower:
   Attitude.—Horizontal.
   Pedicel.—Present.
   Length of pedicel.—Medium.
   Length of flower.—Medium — 2.8 cm.
   Predominant color.—Blue/Turquoise.
   Intensity of predominant color.—Medium.
   Outer perianth segment.—color of basal part just before opening of flower Blue-green (RHS # 121A).
   Outer perianth segment.—color of basal part of fully opened flower Blue-green (RHS # 121A).
   Outer perianth segment.—color of apex relative to other part Clearly different (Purplish).
   Inner perianth segment.—distinct spot in middle of apex Present.
   Inner perianth segment.—color of margin of apex relative to other part Clearly different (Purple).
   Inner perianth segment.—predominant color of exposed part (excluding differently colored margin of apex) Blue-green (RHS # 121A).
   Length of inner perianth segment compared to outer segments.—Longer.
   Extrusion of stamens.—Absent.
   Fragrance.—Absent.
   Bulb.—predominant shape: Oblate.
   Time of flowering.—Early.
   Duration of flowering.—Medium. The variety is not genetically modified and has been tested to be free of Ornithogalum Mosaic Virus.

The invention claimed is:

1. A new and distinct cultivar of Lachenalia named Aqua Lady as illustrated and described herein.
